(19) United States
(12) Patent Application Publication
(10) Pub. No.: US 2019/0240180 A1
(43) Pub. Date: Aug. 8, 2019

(54) MCT-BASED NUTRITION BLEND FOR PROVIDING HEALTH BENEFITS IN COMPANION ANIMALS

(71) Applicant: NESTEC SA, Vevey (CH)

(72) Inventors: Qinghong Li, Chesterfield, MO (US); George Arthur Bailey Wells, St. Louis, MO (US)

(21) Appl. No.: 16/265,180

(22) Filed: Feb. 1, 2019

Related U.S. Application Data
(60) Provisional application No. 62/627,907, filed on Feb. 8, 2018.

(51) Int. Cl. A61K 31/20; A61K 31/202; A61K 31/198; A61K 31/197; A61K 31/355; A61K 33/06; A61K 31/185; A61P 9/12; A23K 20/158; A23K 20/174; A23K 20/142; A23K 20/24; A23K 50/40

(52) U.S. Cl. CPC A61K 31/20; A61K 31/202; A61K 31/198; A61K 31/197; A61K 31/355; A61K 33/06; A23K 50/40; A61P 9/12; A23K 20/158; A23K 20/174; A23K 20/142; A23K 20/24; A61K 31/185

(57) ABSTRACT

A method for reducing blood pressure or treating renal or kidney disease in a companion animal, the method comprising orally administering a composition comprising a therapeutically effective amount of medium chain triglycerides to the companion animal.

### CROSS REFERENCE TO RELATED APPLICATIONS

This application claims priority to U.S. Provisional Application No. 62/627,907 filed Feb. 8, 2018, the disclosure of which is incorporated herein by this reference.

### BACKGROUND

The present disclosure relates generally to food compositions that provide health benefits to a companion animal. More specifically, the present disclosure relates to compositions comprising medium-chain triglycerides and methods comprising administering the compositions to companion animals to provide a health benefit to a companion animal.

Renal failure is one of the most common causes of death in dogs and cats. In animals which suffer from renal disease, several blood indices are used to determine the severity of the disease. These indices include blood urea nitrogen (BUN) and creatinine. BUN and creatinine levels in the bloodstream increase during the course of renal failure because damage to the kidney of the animal makes the kidney inadequate to filter waste products. Because inadequate filtration of waste products is the fundamental basis of renal disease, BUN and creatinine are considered the primary indicators of renal disease.

Other clinical parameters which are important to the animal suffering from renal disease are phosphorus, carbon dioxide, and triglyceride levels. Hyperphosphatemia (abnormally high blood levels of phosphorus) often manifests itself during renal disease. Previous scientific research has indicated that lowering dietary intake of phosphorus is beneficial to lessening the progression of renal disease. However, prior commercial pet food formulations have been unable to provide low levels of dietary phosphorus and still meet all of the amino acid requirements of the canine and feline because the phosphorus component of such diets is primarily derived from ingredients high in protein. Thus, lowering the phosphorus content of the diet required a lowering of the protein components in the diet to levels which are insufficient to supply the amino acid requirements of the animal.

Carbon dioxide levels are an indicator of the level of metabolic buffering (acid-base balance) that occurs in an animal. Metabolic acidosis becomes a problem for an animal suffering from renal disease, and high carbon dioxide levels are indicators of a lack of buffering. Another parameter which is important in animals suffering from renal disease is the blood triglyceride level. It is important in the animal suffering from renal disease as the triglyceride level is often greater than in normal animals. It would be desirable to be able to control these additional parameters in the renal patient through diet.

Current dietary therapies to reduce measured BUN, creatinine, and phosphorus levels include decreasing the amount of dietary protein to levels where amino acids are present in insufficient quantities. Decreasing the level of dietary protein decreases BUN since urea is ultimately derived from protein. However, such diets may result in other problems developing for the animal as the animal's protein needs are unmet. Accordingly, there remains a need in this art for a pet food dietary composition which can provide treatment for renal or kidney disease and associated symptoms.

### SUMMARY

The present disclosure relates to compositions comprising medium-chain triglycerides and methods comprising administering the compositions to a pet to provide a health benefit. More specifically, the present disclosure relates to compositions for a diet which comprises medium-chain triglycerides (MCT) and optionally one or more of omega-3 fatty acids, Vitamin E, magnesium, taurine, lysine, or sulfur-containing amino acids. The present disclosure also relates to methods using the compositions for lowering blood pressure or for treating renal or kidney disease or a condition relating thereto in companion animals.

The present inventors have discovered that an MCT composition can lower blood pressure in companion animals. Such effect can help treat such animals suffering from renal or kidney disease. Accordingly, in a general embodiment, a method of maintaining or improving renal or kidney health in a companion animal is provided. The method can comprise orally administering a composition comprising a therapeutically effective amount of medium chain triglycerides to the companion animal.

In an embodiment, the composition further comprises a component selected from the group consisting of an omega-3 fatty acid, vitamin E, magnesium, taurine, lysine, a sulfur-containing amino acid, and mixtures thereof. The omega-3 fatty acid can be selected from the group consisting of eicosapentaenoic acid (EPA), docosahexaenoic acid (DHA), and mixtures thereof. The sulfur-containing amino acid can be selected from the group consisting of methionine, cysteine, homocysteine, taurine and mixtures thereof.

In an embodiment, the composition can be a complete and nutritionally balanced pet food. In another embodiment, the composition can be a supplement.

In an embodiment, the medium chain triglycerides can be about 0.5 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, or from about 2 wt % to about 10 wt % of the composition.

In an embodiment, the composition can further comprise a component selected from the group consisting of (i) carnitine, (ii) lysine and methionine, (iii) glutathione, and (iv) a mixture thereof.

In an embodiment, the composition has a characteristic selected from the group consisting of (i) a balanced level of magnesium/sodium/potassium, (ii) high protein, and (iii) combinations thereof. In one embodiment, a balanced level of magnesium/sodium/potassium can be provided by a ratio of potassium to sodium from about 5:1 to about 1:1 with the magnesium in an amount of about 0.08 wt % to about 0.25 wt % of the food composition.

In an embodiment, the composition can be administered to the companion animal daily for at least one week.

In an embodiment, the composition can be administered in an amount that provides about 0.001 g to about 50.0 g of the MCTs/kg body weight of the companion animal per day. In one aspect, the amount can provide about 0.1 g to about 5 g of the MCTs/kg body weight of the companion animal per day. In specific aspects, the amount can provide about 0.2 g to about 1.5 g, or even about 0.25 g to about 1 g, of the MCTs/kg body weight of the companion animal per day.

In one embodiment, the companion animal can be a canine.

Additional features and advantages are described herein and will be apparent from, the following Detailed Description and the Figures.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition on a dry matter basis unless expressed otherwise. When reference is made to the pH, values correspond to pH measured at 25° C. with standard equipment.

The terms “food,” “food product” and “food composition” mean a product or composition that is intended for ingestion by an animal and provides at least one nutrient to the animal. The term “pet food” means any food composition intended to be consumed by a companion animal.

The term “companion animal” means a dog or a cat. As used herein, the term “dog” and “canine” can be used interchangeably. In one embodiment, the companion animal can be a canine.

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%. “Kibbles” means pieces of dry or semi-moist pet food which can have a pellet shape or any other shape. Non-limiting examples of kibbles include particulates; pellets; pieces of pet food, dehydrated meat, meat analog, vegetables, and combinations thereof; and pet snacks, such as meat or vegetable jerky, rawhide, and biscuits.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Moreover, the description of some steps as “optional” does not imply that the other steps which are not explicitly described as optional are necessarily required.

Any embodiment disclosed herein can be combined with any other embodiment disclosed herein.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The relative terms “improved,” “increased,” “enhanced” and the like refer to the effects of the composition disclosed herein (a composition comprising a therapeutically effective amount of medium chain triglycerides or a prophylactic dose of medium chain triglycerides) relative to a composition having a lower amount or lacking medium chain triglycerides, but otherwise identical.

The term “therapeutically effective amount” means an amount of a compound of the present invention that (i) treats or prevents the particular disease, condition, or disorder, (ii) attenuates, ameliorates, or eliminates one or more symptoms relating thereto of the particular disease, condition, or disorder, or (iii) prevents or delays the onset of one or more symptoms of the particular disease, condition, or disorder described herein. In one embodiment, therapeutically effect amount can refer to the amount to treat any condition relating to a kidney or renal disease.

A “medium chain triglyceride” is a lipid in which three fatty acids are bound by ester linkages to a glycerol backbone, and at least two and preferably all three of the fatty acids are each between six and twelve carbons in length. The medium-chain fatty acids are caproic acid (comprising six carbon atoms or C6:0), caprylic acid (comprising eight carbon atoms or C8:0), capric acid (comprising ten carbon atoms or C10:0) and lauric acid (comprising twelve carbon atoms or C12:0). In one embodiment, the medium-chain fatty acids are mainly (e.g., at least 98%) in the form of triglycerides. A composition comprising “lipids consisting essentially of medium chain triglycerides” contains medium chain triglycerides as at least 20% of the lipids, in some embodiments at least 30% of the lipids, in other embodiments at least 40% of the lipids, and in some embodiments at least 50% of the lipids in the composition.

### Embodiments

The present disclosure relates to compositions comprising medium-chain triglycerides and methods comprising administering the compositions to a companion animal to provide a health benefit. More specifically, the present disclosure relates to compositions for a diet which comprises medium-chain triglycerides (MCT) and optionally one or more of omega-3 fatty acids, Vitamin E, magnesium, taurine, lysine, or sulfur-containing amino acids. The present disclosure also relates to methods using the compositions for lowering blood pressure or for treating renal or kidney disease or a condition relating thereto in companion animals.

The present inventors have discovered that an MCT composition can lower blood pressure in companion animals. Such effect can help treat such animals suffering from renal or kidney disease. Accordingly, in a general embodiment, a method of maintaining or improving renal or kidney health in a companion animal is provided. The method can comprise orally administering a composition comprising a therapeutically effective amount of medium chain triglycerides to the companion animal. Such methods can further comprise identifying a companion animal with kidney or renal disease, or at risk for kidney or renal disease, or having elevated blood pressure, or at risk for having elevated blood pressure.

The methods generally comprise orally administering to the dog a composition comprising medium chain triglycerides and optionally one or more of omega-3 fatty acids, Vitamin E, magnesium, taurine, lysine, or sulfur-containing amino acids. The composition can be a pet food, such as a wet pet food, a semi-moist pet food, or a dry pet food, e.g., kibble.

Generally, the medium chain triglycerides can be about 0.5 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, or from about 2 wt % to about 10 wt % of the composition. The medium chain triglycerides may be prepared by any known process, such as direct esterification, rearrangement, fractionation and/or transesterification. For example, the medium chain triglycerides may be prepared from a source of vegetable oil, such as coconut oil, through a rearrangement process. The chain length and distribution thereof may vary depending on the source oil. For example, MCTs containing 1-10% 6, 30-60% C8, 30-60% C10 and 1-10% C12 can be derived from palm oil and/or coconut oil; in some embodiments, at least a portion of the MCTs are provided by coconut oil, but in other embodiments the composition does not contain coconut oil. MCTs containing at least about 95% C8 can be made by semi-synthetic esterification of octanoic acid to glycerin; in some embodiments thereof, the remainder of the fatty acids are C6 and C10. Mixtures comprising MCTs with about 50% total C8 and/or about 50% total C10 are also useful herein.

Non-limiting examples of suitable omega-3 fatty acids include eicosapentaenoic acid (EPA), docosahexaenoic acid (DHA), alpha-linolenic acid (ALA) and mixtures thereof. In one embodiment, the omega-3 fatty acids can range from about 0.2 wt % to about 3 wt % of the composition. In some embodiments, the omega-3 fatty acids are at least about 0.2 wt %, at least about 1.0 wt %, or at least about 2.0 wt %.

Non-limiting examples of suitable sulfur-containing amino acids include methionine, cysteine, homocysteine, taurine and mixtures thereof.

In some embodiments, the composition can be administered to the companion animal for a time period of at least one week, at least one month, at least two, three, four, five or six months; and in some embodiments, for at least one year. During the time period, the composition can be administered to the dog at least one day per week, at least two days per week, at least three, four, five or six days per week; or even seven days per week. The composition can be administered in a single dose per day or in multiple separate doses per day. In an embodiment, the composition can be administered in an amount that provides about 0.001 g to 50 g of the MCTs per kg body weight of the companion animal per day. In one aspect, 0.1 g to about 5 g of the MCTs per kg body weight of the companion animal can be administered per day.

In one specific embodiment, the companion animal can be a canine.

In an embodiment, the composition further comprises (i) carnitine, (ii) lysine and methionine, (iii) an antioxidant, such as glutathione, or (iv) mixtures thereof. The composition can be high in protein, for example at least about 20 wt %, at least about 25 wt %, or even at least about 30 wt % of the composition. Additionally, the composition can have balanced amounts of magnesium, sodium and potassium; for example, the ratio of potassium to sodium can be about 5:1 to about 1:1, in one aspect, about 5:1 to about 2:1, with the magnesium in an amount of about 0.08 wt % to about 0.25 wt %, and in one aspect, from about 0.10 wt % to about 0.15 wt %. At least a portion of the magnesium, sodium and potassium can be provided as isolated compounds (e.g., salts). Alternatively or additionally, at least a portion of the magnesium, sodium and potassium can be provided by one or more foodstuffs. For example, magnesium can be provided by wheat bran, whole grains, leafy green vegetables, meat, beans and bananas; and potassium and sodium can be provided by meats, fish, whole grains, yogurt, bananas, sweet potatoes, squash, beans and tomatoes.

The pet food compositions disclosed herein can be any food formulated for consumption by a pet such as a dog. In an embodiment, the pet food composition provides complete nutrition as defined by the Association of American Feed Control Officials (AAFCO) and which depends on the type of animal for which the composition is intended (e.g., a dog). In another embodiment, the composition can be a supplement. Such a supplement can be added to a food composition or be administered in conjunction with a food composition, or administered separately.

The pet food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

The pet food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as starches, humectants, oral care ingredients, preservatives, amino acids, fibers, prebiotics, sugars, animal oils, aromas, other oils additionally or alternatively to vegetable oil, salts, vitamins, minerals, probiotic microorganisms, bioactive molecules or combinations thereof.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

Yet another aspect of the present disclosure is a method of making a pet food, the method comprising adding MCTs to at least one other comestible ingredient, the MCTs are added in an amount effective to prevent or treat renal or kidney health or reduce blood pressure. For example, the MCTs can be added such that a single serving of the pet food comprises an amount of the MCTs effective to prevent or treat renal or kidney health and/or maintain or lower blood pressure. Optionally one or more of omega-3 fatty acids, Vitamin E, magnesium, taurine, lysine, or sulfur-containing amino acids can be included in the pet food.

### Example

By way of example and not limitation, the following non-limiting study is illustrative of compositions and methods using MCTs for controlling or lowering blood pressure or treating kidney or renal disease in a companion animal, in one or more embodiments provided by the present disclosure.

### Example 1—Blood Pressure Study for Canines

Seven Beagles and 1 Miniature Schnauzer, all of which were examined and classified with degenerative mitral valve disease (DMVD) by a board-certified veterinary cardiologist using echocardiography, were selected for the study. Dogs were randomly assigned into two dietary treatment groups. The control diet provides complete and balanced nutrition manufactured by the Nestle Purina PetCare Company to meet or exceed the requirements as defined by the Association of American Feed Control Officials (AAFCO). The test diet contained the same ingredients as in the control diet except an additional 7% medium chain triglycerides (MCTs). The diets are isocaloric and isonitrogenous. The diets are shown in table 1.

TABLE 1

| Ingredients/Nutrients | Control Diet | Test Diet |
|---|---|---|
| Medium chain triglycerides (MCT), % | 0.0 | 7.0 |
| Omega-3 fatty acids, % | 0.8 | 0.8 |
| Lysine, % | 2.3 | 2.3 |
| Methionine + cysteine, % | 2.1 | 2.1 |
| Magnesium, % | 0.15 | 0.15 |
| Vitamin E, IU/g | 0.9 | 0.89 |
| Taurine, % | 0.21 | 0.21 |
| Carnitine, mg/kg | 750 | 750 |
| Sodium | 0.22 | 0.22 |
| Protein | 29.87 | 29.91 |
| Fat | 18.32 | 18.44 |
| Carbohydrates | 36.24 | 36.29 |

Dogs were individually fed to maintain their body weights. Energy intake for maintenance (MER) was estimated using the equation: MER=139*BW^{0.67} (kilocalories), where BW is the body weight of the dog in kilograms. Dogs were weighed weekly and their amount of food offered was increased or decreased by 5% if their BW decreased or increased more than 5% over their initial BW, respectively. Blood pressure was measured at baseline and at 3 months. Dogs received routine healthcare by the staff veterinarians. The results are shown in Table 2.

TABLE 2

| | Systolic Blood | Systolic Blood | |
|---|---|---|---|
| | Pressure Average - | Pressure Average - | |
| Diet | Before Trail | 3 Months | Percent Change |
| Control | 159.93 | 175 | +9.4% increase |
| Test | 185.27 | 166.4 | −10.2% decrease |

As shown in Table 2, canines on the test diet showed an average decrease in systolic blood pressure over the 3 month period vs canines on the control diet which had an average increase in systolic blood pressure.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for reducing blood pressure in a companion animal, the method comprising orally administering a composition comprising a therapeutically effective amount of medium chain triglycerides to the companion animal.

2. The method of claim 1, wherein the composition further comprises a component selected from the group consisting of an omega-3 fatty acid, Vitamin E, magnesium, taurine, lysine, a sulfur-containing amino acid, and mixtures thereof.

3. The method of claim 2, wherein the omega-3 fatty acid is selected from the group consisting of eicosapentaenoic acid (EPA), docosahexaenoic acid (DHA), and mixtures thereof.

4. The method of claim 2, wherein the sulfur-containing amino acid is selected from the group consisting of methionine, cysteine, homocysteine, taurine and mixtures thereof.

5. The method of claim 1, wherein the composition is a complete and nutritionally balanced pet food.

6. The method of claim 1, wherein the medium chain triglycerides are about 0.5 wt % to about 60 wt % of the composition.

7. The method of claim 1, wherein the medium chain triglycerides include a medium-chain fatty acid selected from the group consisting of caprylic acid, capric acid, and a mixture thereof.

8. The method of claim 1, wherein the composition further comprises a component selected from the group consisting of carnitine, lysine and methionine, glutathione, and a mixture thereof.

9. The method of claim 1, wherein the composition has a characteristic selected from the group consisting of (i) ratio of potassium to sodium from about 5:1 to about 1:1, (ii) magnesium in an amount from about 0.08 wt % to about 0.25 wt %, (iii) protein in an amount of at least 20 wt %, and (iv) combinations thereof.

10. The method of claim 1, wherein the composition is administered to the companion animal daily for at least one week.

11. The method of claim 1, wherein the composition is administered in an amount that provides about 0.001 g to about 50.0 g of the MCTs per kg body weight of the companion animal per day.

12. The method of claim 1, wherein reducing blood pressure provides a renal or kidney benefit.

13. The method of claim 1, wherein the companion animal is a canine.

14. A method of treating kidney or renal disease or a condition relating thereto in a companion animal in need thereof, the method comprising orally administering a composition comprising a therapeutically effective amount of medium chain triglycerides to the companion animal.

15. The method of claim 14, wherein the medium chain triglycerides are about 0.5 wt % to about 60 wt % of the composition.

16. The method of claim 14, wherein the composition further comprises a component selected from the group consisting of an omega-3 fatty acid, Vitamin E, magnesium, taurine, lysine, a sulfur-containing amino acid, and mixtures thereof.

17. The method of claim 14, wherein the composition has a characteristic selected from the group consisting of (i) ratio of potassium to sodium from about 5:1 to about 1:1, (ii) magnesium in an amount from about 0.08 wt % to about 0.25 wt %, (iii) protein in an amount of at least 20 wt %, and (iv) combinations thereof.

18. The method of claim 14, wherein the companion animal is a canine.

19. The method of claim 14, wherein the condition is elevated blood pressure.
